(19) United States
(12) Patent Application Publication
(10) Pub. No.: US 2026/0209666 A1
(43) Pub. Date: Jul. 23, 2026

(54) HIGH DAMAGE THRESHOLD FILMS FOR LASER CELL PROCESSING

(71) Applicant: Cellino Biotech, Inc., Cambridge, MA (US)

(72) Inventors: Christopher Harrison, Cambridge, MA (US); Matthias Wagner, Cambridge, MA (US); Matthew Sullivan, Westwood, MA (US); Alexander Stange, Somerville, MA (US); Scott Luro, Somerville, MA (US); Shoshana Das, Cambridge, MA (US)

(21) Appl. No.: 19/455,018

(22) Filed: Jan. 21, 2026

Related U.S. Application Data
(60) Provisional application No. 63/748,800, filed on Jan. 23, 2025.
(60) Provisional application No. 63/773,051, filed on Mar. 17, 2025.

(51) Int. Cl. C12M 1/12

(52) U.S. Cl. CPC C12M 25/04

(57) ABSTRACT

Cell culture systems for use in an optical bioprocess are disclosed. A multilayer film stack for optical cell management in a cell culture container comprises a substrate having a first surface facing an interior of the cell culture container; one or more absorbing layers on the first surface of the substrate; and a biosurface treatment on top of the one or more absorbing layers, wherein cells are adhered to the biosurface treatment.

### CROSS-REFERENCE TO RELATED APPLICATIONS

This application claims the benefit of U.S. Provisional Application No. 63/748,800, filed Jan. 23, 2025, and U.S. Provisional Application No. 63/773,051, filed Mar. 17, 2025, each of which is incorporated herein by reference in its entirety.

### STATEMENT REGARDING FEDERALLY SPONSORED RESEARCH OR DEVELOPMENT

This invention was made with U.S. government support under Agreement No. 1AY2AX000040-01 awarded by the Advanced Research Projects Agency for Health. The U.S. government has rights in the invention.

### BACKGROUND

Cell therapies hold the promise of aiding millions around the world by treating a wide variety of ailments and illnesses. However, to treat patients cell therapy providers must manufacture cells numbering in the hundreds of millions to billions for each patient. Traditionally, cell manufacturing has been conducted manually, by technicians working in labs around the clock for months to generate a sufficient number of cells (that pass quality controls) for just one patient. This method of cell manufacturing is prohibitively expensive and slow, and thus becomes a bottleneck for cell therapy companies to overcome.

Efforts have been made to automate and/or scale up cell manufacturing, but these efforts are still in their infancy. There are many technical challenges that must be overcome. For example, some approaches for adherent cell management in a closed cell culture container involve applying optical pulses (e.g., laser pulses) to image and to manage/remove cells from the cell culture surface. To enable laser-based cell culture monitoring and management, an additional surface or film is applied to the cell culture surface that enables both imaging and laser-based precision cell management. However, fabricating films that combine optical energy absorption for targeted biological effects using heat and or/bubbling is challenging. The films must satisfy several requirements: they should be semitransparent for high-quality imaging; they should be robust such that they can absorb sufficient laser or other energy without degradation or changing its properties; they should not degrade in cell culture conditions; and they should be non-cytotoxic and moreover support adherent or other cell growth, including supporting protein attachments such as extracellular matrix. Thus, there is a need in the art for improved films for use in laser-based cell processing and management.

### BRIEF SUMMARY

The present disclosure relates to a film stack for optical cell management, and more particularly to a multilayer optical structure configured for use in cell culture containers that enables both optical imaging and optical-based removal of adhered cells.

In one aspect, the film stack comprises a substrate having a first surface facing an interior of a cell culture container, one or more absorbing layers disposed on the first surface of the substrate, and a biosurface treatment on top of the one or more absorbing layers to which cells may adhere. The substrate may be transparent. The film stack may be configured to transmit optical energy within a first wavelength range to enable optical imaging of cells adhered to the stack, while also being configured to absorb optical energy within a second wavelength range to enable optical-based removal of those cells.

In certain embodiments, the film stack further comprises an optical layer disposed on a second surface of the substrate, the second surface facing an exterior of the cell culture container. The optical layer may comprise an antireflective coating, a layer that prevents damage to the substrate, and/or a layer that prevents accumulation of debris.

The film stack may further comprise a buffer layer positioned between the substrate and the one or more absorbing layers. The buffer layer may be configured to absorb excess heat from optical energy to prevent damage to the substrate, to tune reflection, transmission, and/or absorption characteristics of the stack, and/or to act as a barrier between the substrate and the one or more absorbing layers.

The one or more absorbing layers may be configured to convert optical energy to mechanical energy or heat energy that is transferred to one or more cells adhered to the stack. This mechanical energy or heat energy may cause the cells to detach from the biosurface treatment, or may cause sections of the biosurface treatment to detach from the one or more absorbing layers. In certain embodiments, the one or more absorbing layers comprise alternating layers of a first material and a second material, wherein the first material absorbs optical energy within a second wavelength range and the second material does not absorb optical energy within the second wavelength range. The layers of the first material may be thinner than the layers of the second material, and the layers of the second material may be of non-uniform thickness. The first material may comprise amorphous silicon, amorphous germanium, amorphous silicon-germanium, gold, or silver. The second material may comprise silicon dioxide, silicon nitride, aluminum oxide, hafnium oxide, yttrium oxide, magnesium fluoride, zirconium oxide, or titanium dioxide.

In further embodiments, the film stack includes a capping layer disposed on top of the one or more absorbing layers. The capping layer may serve as a barrier between a biological volume in the cell culture container and the one or more absorbing layers, may aid in adhering cells and/or proteins to the stack, may prevent degradation of the stack into the biological volume, and/or may provide a biocompatible, non-cytotoxic surface exposed to the biological volume.

The biosurface treatment may be configured to encourage cell growth and adhesion, to withstand high temperatures, and/or to prevent leaching of materials from the stack into a biological volume. In certain embodiments, the biosurface treatment comprises an extracellular matrix.

In another aspect, a cell culture container comprising the film stack described herein is provided.

### BRIEF DESCRIPTION OF THE DRAWINGS

FIG. 1 is a block diagram of a cell culture system in accordance with various implementations.

FIGS. 2A-D are diagrams of a thin film for use in an optical bioprocess in accordance with various implementations.

FIG. 3 is a diagram of a film for use in a cell culture container in accordance with various implementations.

FIG. 4 is a diagram illustrating various physical interaction modes of optical energy absorption on a semitransparent film in a cell culture container in accordance with various implementations.

FIG. 5 is a diagram illustrating a number of functions that the physical interaction modes depicted in FIG. 2 may perform in a biological volume in a cell culture container in accordance with various implementations.

FIG. 6 is a diagram illustrating a biosurface stack for use in a cell culture system in accordance with various implementations.

FIG. 7 are diagrams showing examples of tuning of an absorber structure for optical and thermal performance in accordance with various implementations.

These and other features of the present implementations will be understood better by reading the following detailed description, taken together with the figures herein described. The accompanying drawings are not intended to be drawn to scale. For purposes of clarity, not every component may be labeled in every drawing.

### DETAILED DESCRIPTION

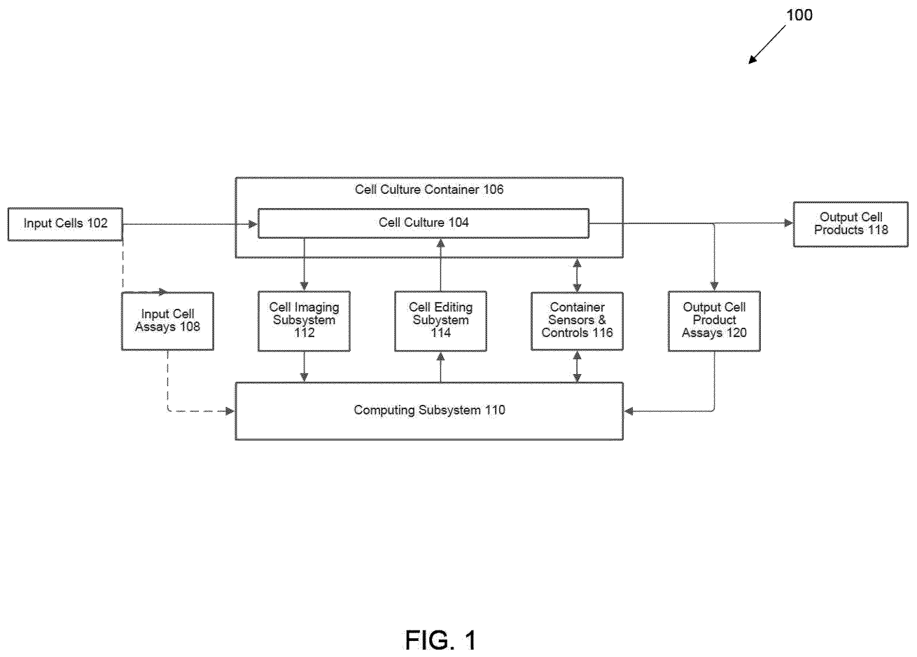

FIG. 1 is a block diagram of a cell culture system 100 in accordance with various implementations. The cell culture system 100 receives input cells 102 as “source” cells upon which the cell culture system 100 performs various cell culture processes. The input cells 102 may be sorted, expanded, or otherwise modified prior to the cell culture performed by the cell culture system 100. Input cell types may include, but are not limited to, somatic cells including but not limited to fibroblasts, mature blood and progenitor cells, such as CD34+ cells and erythroblasts, keratinocytes, epithelial cells, including blood and urine-derived epithelial cells, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other muscle cells, and generally any live somatic cells. The term “somatic cells,” as used herein, also includes adult stem cells and pluripotent stem cells (including but not limited to induced pluripotent stem cells and embryonic stem cells).

The input cells 102 may be analyzed with one or more input cell assays 108 which serve to quantify the state of the input cells 102. The input cell assays 108 may be nondestructive (such as cell counting) or a sample may be extracted for tests including, but not limited to, genomic profiling, gene expression assays such as PCR, qPCR, microarray, single-cell RNA sequencing, whole exome sequencing (WES), whole genome sequencing (WGS), karyotyping, short tandem repeat (STR) analysis, sterility testing (testing for bacteria and viruses), or other phenotype analysis including, but not limited to, cell surface antigen or intracellular staining-based immunofluorescence or flow analysis, and cell viability, morphology and migration assays, or any other implementations known to persons of ordinary skill in the art. The sample extraction can be performed using automated or semi-automated processes within a closed cell culture environment to enable continued propagation of the cell culture within a sterile environment. The results of these assays are transmitted to a computing subsystem 110, which may use the results in various software applications to monitor, predict, and control the cell culture process performed by the cell culture system 100.

The input cells 102 are placed into a cell culture 104, where they will remain for the duration of the processes performed by the cell culture system 100. The cell culture 104 may reside in a cell culture container 106. The cell culture container 106 may include one or more chambers to hold the cell cultures, and may take the form of microwell plates, flasks, stackable cell culture containers, closed cassette systems, microfluidic chambers, purpose-built bioreactor vessels, or any other implementations known to persons of ordinary skill in the art. The cell culture container 106 may be a closed/sealed sterile environment for the cell culture 104 and fluid media used in cell culture processes. The cell culture container 106 may include a cell culture chamber having a first surface upon which cells may adhere. The first surface may be semi-transparent to enable optical-based cell monitoring and manipulation. For example, the first surface may be configured to transmit optical pulses within certain wavelength ranges to enable optical imaging of the cells adhered to the first surface. The first surface may also absorb optical within other wavelength ranges to enable cell removal or cell manipulation functions. For example, the first surface may be configured to convert optical energy into heat or mechanical energy in the form of microbubbles to detach and/or kill cells adhered to the first surface. Compositions of the first surface are described herein.

The cell culture 104 may be used for a number of cell processes performed and monitored by the cell culture system 100, including but not limited to: cell reprogramming (into pluripotent or multipotent forms), cell differentiation, cell trans-differentiation, cell expansion, cell sorting, clonal isolation, cell gene editing, cell-based protein production, cell-based viral production, cell rejuvenation or regeneration, combinations thereof, or any other implementations known to persons of ordinary skill in the art.

The cell culture container 106 may be in a format that allows for observation of the cell culture 104 at regular intervals using an imaging subsystem 112. For example, the cell culture container 106 may include a closed cassette system having at least one transparent or semi-transparent surface that allows for light or laser-based imaging and editing. The imaging subsystem 112 may be configured to provide label-free imaging suitable for long-term cell culture observation, although some implementations may include fluorescent imaging capability for immunofluorescent or other labeled images. Label-free modalities employed by the imaging subsystem 112 may include, but are not limited to, brightfield imaging, phase imaging, darkfield imaging, transmission imaging, reflection imaging, quantitative phase imaging, holographic imaging, two-photon imaging, autofluorescence imaging, Fourier ptychographic imaging, defocus imaging, or any other implementations known to persons of ordinary skill in the art. The imaging subsystem 112 may be shared between one or more of the cell culture containers 106.

The cell culture system 100 further includes a cell editing subsystem 114 for editing the cell culture 104. The cell editing subsystem 114 may edit the cell culture 104 at a regional, colony-specific, and/or cell-specific level. Editing, in this context, may include selective destruction and/or removal of cells or cell regions, and non-destructive operations on cells (including intracellular delivery of compounds into cells or extraction of compounds from cells). The cell editing subsystem 114 may edit the cell culture 104 through a variety of directed energy mechanisms. In other words, the cell editing subsystem 114 may generate energy that is directly used to edit cells and/or converts energy of one form (e.g., light, mechanical) into energy of another form to achieve cell editing. The mechanism by which the cell editing subsystem 114 acts upon cells in the cell culture may include, but not be limited to, robotic systems that mechanically actuate a tip or tool across the cell culture, magnetic actuators in conjunction with magnetic tools that interact with the cell culture, systems that are configured to selectively apply an electric field across portions of the cell culture, ultrasound systems that are configured to apply ultrasonic energy to portions of the cell culture, droplet or particle ejection/acceleration systems that are designed to impact droplets or particles on portions of the cell culture, optical systems that are designed to deliver optical energy to portions of the cell culture, combinations thereof, or any other implementations known to persons of ordinary skill in the art. The cell editing subsystem 114 may be shared between one or more of the cell culture containers 106.

Optical mechanisms for cell editing may include, but are not limited to, optical systems that direct energy directly into cells or surrounding media in the cell culture, optical systems that direct energy into particles or dyes that are added to the cell culture media (including, but not limited to particles functionalized in a manner to attach to specific cells, or that are taken up by cells), or optical systems that direct energy into particles or films that are on surfaces proximate to portions of the cell culture, or any other implementations known to persons of ordinary skill in the art. Optical mechanisms may operate on the cell culture by a number of approaches including, but not limited to, elevating the local temperature to a point where cells are destroyed due to heat damage, elevating local temperature to cause boiling and/or bubble formation to cause portions of the cell culture to detach from a surface, or elevating local temperature rapidly in order to cause rapid bubble formation and then subsequent collapse to affect mechanical forces on the local cell membranes, or combinations thereof.

The cell culture system 100 may also include a number of sensors and controls 116 which may measure or act upon the cell culture 104. For example, the sensors and controls 116 may carry out functions such as measuring media conditions within the cell culture 104, causing fresh media to be supplied, or adding reagents or gases in order to adjust media conditions for optimal cell culture growth. Sensors that sense the state of the cell culture 104, cell culture media, and/or surrounding cell culture container 106 may include, but are not limited to, temperature sensors, humidity sensors, gas composition sensors including but not limited to O₂ and CO₂ concentration sensors, gas flow rate sensors, dissolved gas sensors including but not limited to dissolved O2 sensors, liquid flow rate sensors, and sensors to measure cell culture media constituents (such as nutrients, waste products, vitamins, metabolites, proteins, extracellular vesicles, cell mass, or cell debris) including but not limited to optical absorption sensors, optical scattering sensors, mass spectroscopic sensor systems, optical or electrical pH sensors, and viscosity sensors.

Controls that may interact with the cell culture 104 or the cell culture container 106 may include, but are not limited to, liquid handling systems that inject or extract various liquids to/from the cell culture 104 or the cell culture container 106, environmental control systems that control the temperature or other environmental parameters of the cell culture 104 or the cell culture container 106, power systems that provide electrical power to the cell culture container 106, and mechanical or robotic systems that may move or manipulate the cell culture container 106 or portions thereof.

The computing subsystem 110 may be configured to control the other components of the cell culture system 100 to perform the specified cell culture process on the cell culture 104 to produce output cell products 118. The output cell products 118 may include both cells and cell-derived products, and may be harvested from the cell culture 104. Output cell products 118 that may be produced by the computing subsystem 110 may include, but are not limited to, induced pluripotent stem cells, differentiated cells (e.g., differentiated from iPSCs), proteins (e.g., cytokines, antibodies, hormones), lipid particles (e.g., exosomes), viral particles, somatic cells (including but not limited to fibroblasts, mature blood and progenitor cells, such as CD34+ cells and erythroblasts, keratinocytes, epithelial cells, including blood and urine-derived epithelial cells, Sertoli cells, endothelial cells, granulosa epithelial, neurons, pancreatic islet cells, epidermal cells, epithelial cells, hepatocytes, hair follicle cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, other muscle cells, generally any live somatic cells, and the combination of any of the above. The term “somatic cells,” as used herein, also includes adult stem cells.

The output cell products 118 may be measured by output cell product assays 120 in order to determine critical product parameters such as phenotype distribution, protein production, gene activation, genomic makeup (including but not limited to genomic profiling assays such as PCR, qPCR, microarray, single-cell RNA sequencing, whole exome sequencing (WES), whole genome sequencing (WGS), karyotyping, short tandem repeat (STR) analysis, sterility testing (testing for bacteria and viruses)), or other phenotype analysis including but not limited to cell surface antigen or intracellular staining and immunofluorescence or flow analysis and cell viability, morphology and migration assays, or potency assays such as self-renewal and teratoma formation assays, and germ-layer differentiation assays. The output assay data may be conveyed to the computing subsystem 110 in order to refine predictive models (based on image data, sensor data, information from prior cell culture processes, and other information sources) for cell culture monitoring and control. Output cell product assays 120 may include, but are not limited to, viability assays, cell counting, flow cytometry, immunostained imaging assays, PCR assays (including but not limited to qPCR and/or ddPCR), RNA sequencing assays including single-cell RNA assays, cell differentiation assays, embryoid body formation assays, trilineage differentiation assays, karyotyping assays, DNA sequencing, or any other implementations known to persons of ordinary skill in the art.

The computing subsystem 110 is configured to gather data from a range of sources, organize the data in a manner that allows it to make predictions of success/quality/functionality of the cell culture 104, and in many cases do so on a cell-by-cell, colony-by-colony, or region-by-region basis. For example, using local cell density and proliferation rate data obtained through analysis of the time series of label-free images provided by the imaging subsystem 112, in conjunction with data regarding the input cells (in order to control for patient-specific factors, for instance), and based on a large number of observed histories and corresponding cell quality data measured by the output cell product assays 120, the computing subsystem 110 may predict which regions of cells are most likely to yield superior cell products, and which regions are less likely to yield good product. In situations where cell media is limited or there is competition between cells for space in the cell culture container 106, the computing subsystem 110 may instruct the cell editing subsystem 114 to remove the regions or even individual cells predicted to underperform.

Another function of the computing subsystem 110 is to use cell data derived from imaging in conjunction with environmental parameters and sensor data from the sensors and controls 116 and assay data from the input cells 102 and/or the output cell products 118 in order to pre-emptively adjust cell culture conditions according to cell count, proliferation rate, differentiation status, phenotype, or other factors in addition to real-time cell media readings. Using a model trained on previous iterations, the computing subsystem 110 may adjust media conditions such as fresh media feed, media type, temperature, pH, dissolved oxygen levels, reagent or vitamin levels or other global cell culture properties using the controls 116. Similarly, the computing subsystem 110 may use cell data obtained from imaging, potentially in conjunction with cell media sensor data, to determine when the cell culture 104 is ready for harvest. Actuators utilized by the controls 116 may include, but are not limited to: liquid handling robots, liquid circulation systems including valves and pumps, temperature control elements, pH controllers, gas exchange mechanisms to control dissolved gases or any other implementations known to persons of ordinary skill in the art.

The computing subsystem 110 may control the cell editing subsystem 114 to make edits to the cell culture 104 according to cell management algorithms (for example, to maintain a certain cell density, to maintain certain exclusion areas within the cell culture container), in a timed manner (for example, delivering gene-activating or gene-editing compounds to cells at a specific interval), and/or as a result of predictions made by the computing subsystem 110 (for example, removal of cells predicted not to yield the desired phenotype or optimal level of function). “Editing” may include both destruction of cells and/or colonies (including inducing apoptosis, lysing, physically removing), as well as selective delivery of compounds into cells and/or regions of cells via intracellular delivery mechanisms, or selective extraction of compounds from the cells via intracellular delivery mechanisms, or other types of cell manipulation.

The computing system 110 may include elements that perform conventional image processing (including but not limited to filtering, normalization, contrast enhancement, z-stack processing, thresholding, histogram transformations, edge detection, correlations, convolutions, frequency space operations, blob detection, morphological operations, registration, warping, object detection, object tracking or combinations thereof), deep learning based image processing (including but not limited to convolutional neural networks, fully-connected neural networks, semantic and instance-level segmentation, encoder-decoder networks, multi-scale algorithms, recurrent networks, visual attention models, vision transformers, generative adversarial models, U-Nets, ResU-Net, SegNet, X-Net, ENet, BoxENet, long short-term memory neural networks, and combinations thereof), statistical models, pattern recognition, statistical learning (including but not limited to linear regression, non-linear regression, hierarchical regression, generalized linear models, logistic regression, log-linear models, non-parametric models), machine learning (including but not limited to decision trees, random forest, support vector machines, neural nets, deep learning, association models, sequence modeling, genetic modeling), clustering techniques including hierarchical and non-hierarchical clustering, supervised machine learning models, unsupervised machine learning models, databases (including but not limited to SQL databases and NoSQL databases), visualization tools for image, cell, colony, clone and other data, combinations of these elements, or any other implementations known to persons of ordinary skill in the art.

The computing subsystem 110 may also include data storage for storing image data, sensor data, the results of data analysis, and program code that the computing subsystem 110 executes. The computing subsystem 110 may also include input/output devices to allow users to view data and monitor and control the cell culture system 100, or to transfer data in and out of the cell culture system 100. For example, the computing subsystem 110 may include display screens, monitors, communications/interface ports, keyboards, audio systems, and the like. The computing subsystem 110 may be proximate to the other components in the cell culture system 100 (e.g., a local computer) or may be remote from the other components in the cell culture system 100 (e.g., a cloud server). In some implementations, the computing subsystem 110 may have one or more components proximate to the other components in the cell culture system 100 and some components remote from the other components in the cell culture system 100. The computing subsystem 110 may be configured to communicate with the other components in the cell culture system 100 utilizing a wired and/or wireless connection (e.g., Ethernet cables, optical fiber, Wi-Fi, Bluetooth), and may be configured to communicate with external components utilizing a wired and/or wireless connection. The computing subsystem 110 may have additional functionality and components not disclosed herein, but would be apparent to a person of ordinary skill in the art.

The cell culture system 100 may be configured to allow extended cell culture processes to be performed within a single cell culture container 106 using the cell editing subsystem 114. Because the cell editing subsystem 114, as directed by the computing subsystem 110, can selectively remove cells from cell culture, the cell culture does not overgrow the cell culture container, and therefore does not require frequent transfers (“passaging”) which are stressful on cell populations, disrupt cell processes, introduce potential sterility and contamination issues, and make time series tracking of cell-, region-, colony- or clone-specific behavior impossible. Thus, the combination of continuous monitoring via image and sensor data—enabled by the single-container process—may allow the computing subsystem 110 to predict the optimal regions or cells to remove to maintain low enough cell density to remain in the single cell culture container 106. In the process the cell culture system 100 may also perform in-place “sorting” of cells to enrich the population according to real-time measurements.

### Thin Film and Laser Specifications

The cell culture surface of a cell culture container may include a thin optical film to facilitate optical-based imaging and cell manipulation (e.g., removal from the cell culture surface). The thin film and the optical light source (e.g., a laser) should meet certain specifications or constraints to effectively remove target cells without damaging other cells and having minimal degradation of the film itself over time. To generate microbubble cavitation on the cell culture surface, energy is absorbed rapidly in a tightly confined volume. Cells and cell media are transparent in the visible region of the spectrum, but have strong absorption lines in the ultraviolet (UV) and mid-infrared (mid-IR). However, UV light is avoided to prevent ionizing radiation and the high cost and power issues presented by the appropriate mid-IR lasers. Femtosecond and tightly confined nanoseconds lasers in the visible and near-IR can induce dielectric breakdown in media, but this process is difficult to precisely and reliably control. More cost-effective nanosecond lasers in the visible and near-IR offer sufficient power to generate sufficiently rapid heating but require an absorbing substrate (i.e., a thin film).

Effective methods for cell killing requires sufficient energy that the bubble be of similar size as the cells. For example, for a cell killing threshold of 200 nJ (roughly 60 nJ absorbed) for well-separated spots (8 microns) at 100 kHz, a maximum bubble diameter of 16 microns of lifetime 2-3 microseconds has been measured. The energy to heat water from 37° C. (typical cell culture temperature) to 100° C. and supply the latent heat for the phase change, assuming a hemispheric bubble, is calculated at an energy of 1.6 nJ, a factor of 40 lower than the optically absorbed energy. Various factors determine this higher supplied energy, such as the thermal diffusivity of glass being almost three times higher than that of water (wicking heat away), the energy dissipated by the shock wave, and any inefficiency in energy transfer from the film to the liquid due to vapor being nucleated early during the 20 nanosecond laser pulse.

For laser light to be absorbed and locally nucleate a bubble, the glass on which cells are grown must either be stained or coated. Inconel, a nickel-based alloy, has been popularized for use in neutral density filters, both for non-reflective (volumetric, like stained glass) and reflective types, the latter consisting of a thin coating. The laser induced damage threshold (LIDT) limit for one example commercially available unit is 0.025 J/cm². The 200 nJ used for killing cells translates to a peak free space fluence of 0.16 J/cm², or an optically absorbed fluence in glass of 0.072 J/cm² (far above the thin film commercially available ND filter), illustrating the challenging optical film requirements. The damage threshold increases if a laser pulse's energy can be distributed in time and space, for example by increasing the pulse duration from 3 to 20 nanoseconds and increasing the free space beam width (“D4σ”) to no larger than 18 microns (or 12 microns when focused in glass).

The thin film thickness may have a thickness of a few hundred nanometers to spread heat in the plane normal to the surface of the thin film. A thin film suitable for an all-optical bioprocess should be configured to withstand multi-month immersion in a warm saline environment (e.g., cell culture media) as well as withstand laser pulses. Since the thermal diffusion distance in glass is on the order of 80 nanometers during a 20 nanosecond pulse, in some implementations the film may include alternating layers of absorbing and transparent films, with an inter-layer spacing of 30 nm or less. In some implementations, the transparent layer may be silicon or titanium oxide, and the absorbing layer may be gold or amorphous silicon. In some implementations, the film may be protected by an even thinner layer of titanium dioxide.

### Rugged Thin Films

Optically absorbing thin films coated onto glass coverslips immersed in cell culture media can be exposed to ~10 nanosecond laser pulses to generate cavitation events which kill adherent cells. The preferred spot size for cell editing is on the order of a few microns since this permits the targeting and killing of individual cells. However, localized laser-based heating of thin films can result in film damage when fluence levels are sufficiently high so as to provide cell-killing cavitation events. Furthermore, the harsh environment of continuous immersion in warm cell culture media for months provides an additional challenge for optical films (e.g., corrosion, dissolution). The systems and methods disclosed herein include a thin film design which distributes the heat perpendicular to the film surface, reducing the peak thermal gradient, yet still enables cell destruction.

Aspects of the implementations disclosed herein include (1) that an optically absorbing thin film may maintain integrity when immersed in cell culture media at 37° C. for months without leaching materials into the cell culture media that could lead to cytotoxicity; (2) that a thin film may maintain a high enough surface temperature to create bubbles when in contact with cell culture media which in turn cavitate sufficiently aggressively to kill cells while not self-destructing from cavitation damage; and (3) that a thin film may be configured to facilitate cell growth due to lack of cytotoxicity and low dissolution rate.

Stem cell culture is considered “adherent” because the cell growth occurs when adhered to a surface, preferably coated with “extra-cellular matrix” (ECM) to promote cell health and proliferation. The materials used for the thin film should be carefully selected because stem cells are known to be strongly sensitive to their environment. For example, the thin film may include inorganic materials such as metal oxides or metal nitrides. The composition of the thin film should also encourage binding between the film and the ECM to facilitate cell growth. A further requirement is that the film resist corrosion or chemical degradation from long-term cell culture media (e.g., 60 days at 37° Celsius).

In various implementations, the thin film may also be configured to distribute heat imparted by the laser normal to the film surface while not diffusing far enough laterally to lose the spatial (lateral) resolution of the cell removal technique. Furthermore, the encapsulated optical absorber in the thin film may be configured to not be exposed directly to the cell culture media nor to the cells, even after being exposed to cell-killing laser energy, because most optical absorbers are detrimental to cell growth.

### High Damage Threshold Semi-Absorbing Laser Films

For the purpose of producing a semi-absorptive film that absorbs nanosecond pulsed laser energy and transmits the captured radiation as heat to an adjacent liquid layer, amorphous silicon is a potential candidate because it is non-toxic, may be deposited by various means, and is absorptive at the 532 nm pulsed laser wavelength but more transparent at longer wavelengths (for example at 635 nm) at which live cell imaging may be performed with minimal cell health impact. Further, the absorption coefficient of amorphous silicon is such that relatively thin layers (e.g., on the order of tens of nanometers) provide sufficient energy absorption at 532 nm (absorption coefficient on the order of 1×10⁵/cm). An overall thin coating may be used, which increases the efficiency of rapid energy transfer from absorbed optical energy to cell media for the purpose of triggering and expanding microbubbles. However, high peak temperatures during pulsed laser operation may recrystallize the amorphous silicon layer(s), which leads to a rapid reduction in absorption at 532 nm (towards an absorption coefficient of crystalline silicon of roughly 1×10⁴/cm) and may further cause stresses at film layer boundaries. For example, in some amorphous silicon films, annealing has been shown to shift the film from a 200 MPa compressive stress as deposited to a 1000 MPa tensile stress after recrystallization. In a multi-layer structure that may include spacer films as well as capping films, such shifts in stress may cause cracking, bubbling and/or delamination of the films.

The implementations disclosed herein include a semi-absorptive film comprising a blend of amorphous silicon and silicon dioxide (or silicon nitride) that absorbs sufficient optical energy from 532 nm nanosecond pulses to trigger microbubble formation, but resists recrystallization of amorphous silicon, and the accompanying film stress mismatches. Another potential source of degradation to the absorbing layer is oxidation. Silicon dioxide and titanium dioxide do not form strong oxygen diffusion barriers and may be a source of oxygen above and beyond that in the atmosphere. Thus, silicon nitride layers may function as a good oxygen diffusion barrier to protect the absorbing layers.

Another implementation would be a semi-absorptive film comprising a blend of amorphous silicon and silicon nitride that absorbs sufficient optical energy from 532 nm nanosecond pulses to trigger microbubble formation, but resists recrystallization of amorphous silicon and the accompanying film stress mismatches. Further, silicon nitride provides a superior thermal diffusivity to silicon dioxide and titanium dioxide, which are other candidates for “spacer” and capping layers with amorphous silicon. Moreover, the coefficient of thermal expansion (CTE) of silicon nitride is better matched to amorphous silicon, potentially reducing stress mismatches during rapid heating from laser pulses. Silicon nitride can withstand high temperatures (resisting oxidation up to 1400° C.), is somewhat resistant to dissolution in water including salt water, and forms a good diffusion barrier, including to oxygen to prevent oxidation of encased layers.

Several designs for the semi-absorbing film are possible using the combination of silicon nitride, amorphous silicon, and blends of the two (“silicon-rich silicon nitride”). These materials may be deposited by methods including but not limited to: plasma-enhanced chemical vapor deposition (PECVD); reactive, magnetron, AC or DC sputter deposition using a silicon target and nitrogen-rich plasma; and ion beam assisted e-beam evaporation of silicon, with a nitrogen environment. Generally having separate precursors for the nitrogen and silicon components, and reacting the two during deposition, affords greater flexibility in the design of the coatings under the present implementations.

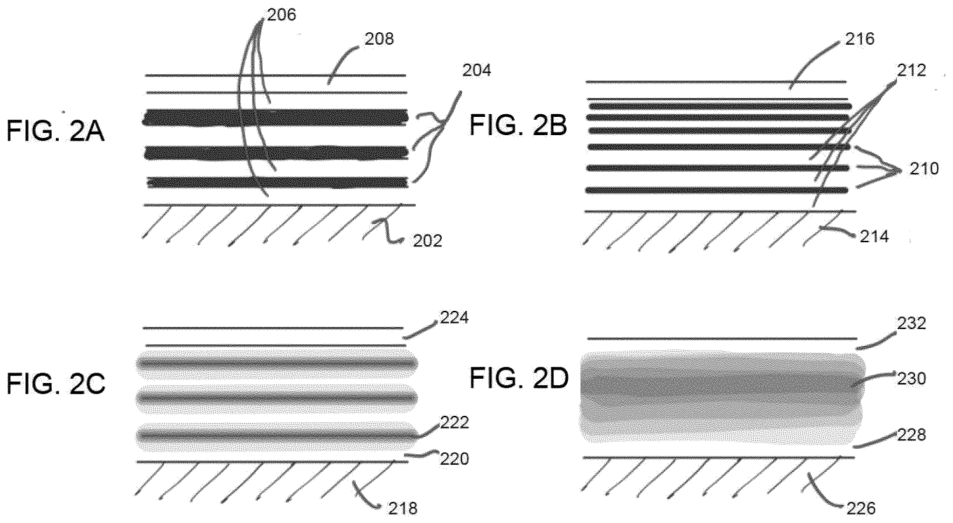

FIGS. 2A-D are diagrams of a thin film for use in an optical bioprocess in accordance with various implementations. FIG. 2A depicts integration of silicon nitride into a distributed-absorber laser film. The film may be deposited onto a transparent substrate 202, which may for example be borosilicate glass, fused silica, or other materials that are clear in the visible wavelength range. The film may include absorbing layers 204 that are made from a material that absorbs at the laser wavelength, for example amorphous silicon to absorb at 532 nm. The film may also include “spacing” layers 206 made from silicon nitride, which has a similar CTE to amorphous silicon, and very good adhesion to amorphous silicon as well as to most substrate materials. In some implementations, silicon nitride also forms a very good diffusion barrier that prevents intrusion of oxygen or other atoms into the structure, including prevention of ion migration from the substrate into the absorber layers 204 where they may speed up recrystallization at high temperature. An additional capping layer 208 may be used as the interface to cell media, extracellular matrix, and cells. In some implementations, the capping layer 208 may be formed from silicon nitride as well. In other implementations, the capping layers 208 may be composed of materials including but not limited to titanium dioxide, silicon dioxide, silicon oxynitride, etc. Silicon nitride also provides higher thermal diffusivity, allowing more efficient conduction of heat from the absorber layers 204 to the biological interface. As indicated in the example shown in FIG. 2A, the thicknesses of the absorbing layers 204 may vary as a function of depth in the film, to account for optical and thermal effects.

FIG. 2B depicts a film having absorbing layers 210 deposited with silicon nitride spacers 212 deposited onto substrate 214, in which the layers are kept very thin to increase the temperature at which the absorbing layers recrystallize. The absorbing layers 210 may be fabricated from amorphous silicon, for example, or amorphous silicon carbide. Keeping the individual absorbing layers to ≤20 nm, ≤10 nm, ≤5 nm or ≤2.5 nm may increase surface-energy effects from the interfaces to silicon nitride, and confinement effects within the absorbing layer, to significantly increase the recrystallization temperature, making the entire structure capable of handling higher peak temperatures. This may also allow an overall thinner structure, which improves efficiency of energy transport from the absorbing layers 210 to a top biological layer 216. As shown, the spacing between absorbing layers 210 may be varied in the structure to optimize optical and thermal properties, for example to equalize the peak temperature experienced by the individual absorbing layers 210 when illuminated with laser pulses from the transparent substrate side. In addition to the spacing between the layers, the thickness of individual layers may be tuned to either make the temperature more uniform or bias the heating to the liquid-film surface.

FIG. 2C depicts shows a semi-absorbing film on substrate 218 that makes use of a continuous range of materials between silicon nitride 220 and silicon-rich silicon nitride 222. Silicon-rich silicon nitride has optical properties, including absorption, that range between pure silicon nitride and amorphous silicon. By creating absorbing structures where there is a continuous gradient between the materials, the film structure may become more resistant to delamination or cracking. By controlling the nitrogen content, recrystallization may be avoided entirely up to very high temperatures. A capping layer 224 may be applied that has good resistance to dissolution in cell media, is biocompatible, and is compatible with cell and/or extracellular matrix adherence. The capping layer 224 may be composed of, for example, silicon nitride.

FIG. 2D depicts a simplified structure taking advantage of the ability to deposit silicon-rich silicon nitride films that offer tunable absorption, high peak operating temperature, and high thermal diffusivity. In this example, a continuous-growth film is deposited onto substrate 226, with initial pure silicon nitride 228, then onto a gradient to silicon-rich silicon nitride 230 which acts as the absorber, and finally onto pure silicon nitride 232 which acts as a thermal conductor, interface to biology, and diffusion barrier. The example shown in FIG. 2D uses a graded material. In other examples a homogeneous absorbing layer of silicon-rich silicon nitride may be used. In some implementations, germanium may be used to suppress crystallization of amorphous silicon. Thus, the film structures described with reference to FIGS. 2A-D have a top surface that is compatible with long-term cell culture conditions, ECM attachment, and healthy cell growth, while providing the laser-initiated rapid bubble formation and collapse necessary for cell processing.

### Thin Films Optical Bioprocess

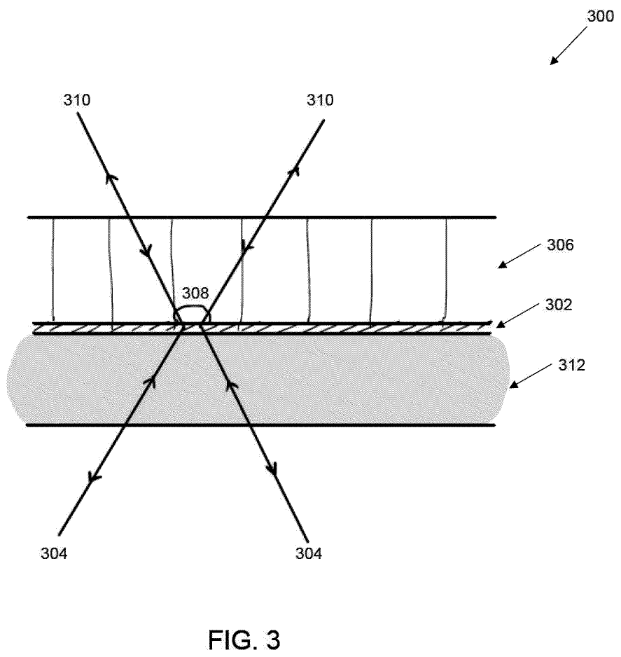

FIG. 3 is a diagram of a film for use in a cell culture container 300 in accordance with various implementations. The cell culture container 300 includes a semitransparent film 302 that absorbs optical radiation 304, leading to an effect within a biological volume 306 in the cell culture container. The biological volume 306 may include cells, media, reagents, proteins, extracellular matrices (ECMs), etc. The energy effect of the optical radiation 304 within the biological volume 306 is localized near the location of incidence of the optical radiation 304. This localized effect 308 is caused by the presence of the film 302 adjacent to the biological volume 306. Optical energy may pass through the biological volume 306 before or after interacting with the film 302, without strong absorption in the biological volume 306, as indicated by transmitted rays 310. In this manner, the absorbed energy maximally achieves the desired localized effect 308. The optical radiation 304 may additionally pass through a transparent substrate material 312. The semitransparency of the film 302 allows imaging within the biological volume 306, including but not limited to brightfield, phase, transmission, fluorescent or other imaging approaches. In some implementations, the area of the film 302 illuminated by optical radiation 304 to achieve the localized effect 308 may be kept small to deliver a precise spatial effect on the biological volume. For example, the area may be ≤1 E⁶ μm², ≤1 E⁵ μm², ≤1 E⁴ μm², ≤1 E³ μm², ≤1 E² μm², or ≤1 E¹ μm² in area.

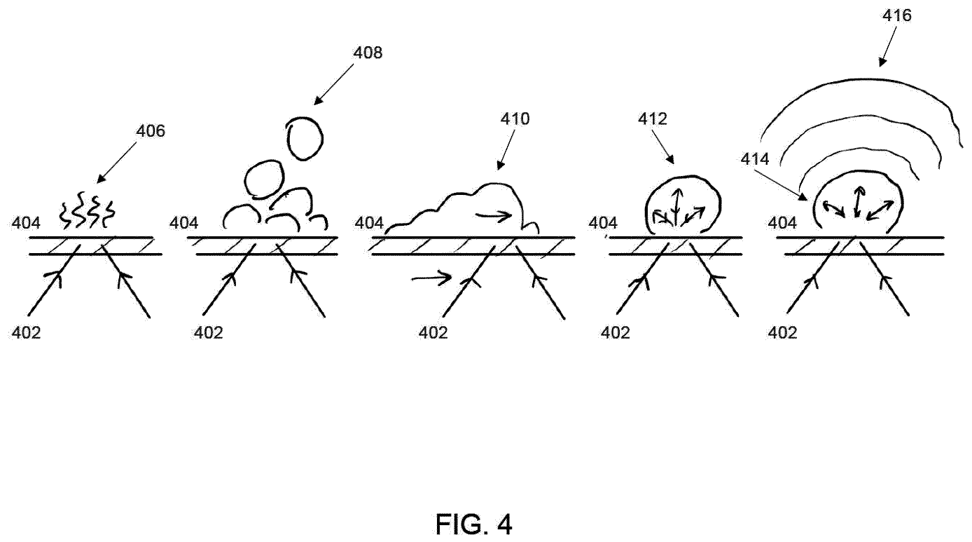

FIG. 4 is a diagram depicting various physical interaction modes of absorption of optical energy 402 on a semitransparent film 404 (e.g., the film 302 in FIG. 3) in a cell culture container in accordance with various implementations. The different physical interaction modes each produce different localized effects in a biological volume of the cell culture container. In one example, the optical energy may be dissipated as heat 406. In another example, the optical energy may be used to cause continuous boiling/bubbling 408. In another example, the energy may be used to generate continuous bubble formation 410 (leading edge) and collapse (trailing edge) that is translated through the biological volume. In another example, the optical energy may be used to trigger the rapid formation, expansion, and collapse of a bubble 412 which exerts mechanical forces directly on surrounding material (including but not limited to cells, tissue, debris, ECM, adsorbed proteins, etc.). In another example, the optical energy may be used to trigger formation, expansion, and collapse of one or more bubbles 414, and the resulting shock waves 416 exert mechanical forces on objects within the biological volume that are not in direct contact with the bubble(s) 414. In the case of single bubble formation, expansion, and collapse, to directly porate and/or kill cells the size of the bubble may be controlled to affect a region approximately the size of a cell. For example, the optical energy may be tuned to produce bubble diameters of ≤50 um, ≤25 um, ≤15 um, or ≤10 um. In some implementations, such bubbles may have short durations such that bubbles may be formed and collapsed independently in a small area, but with rapid throughput. For example, the bubbles may have durations of ≤25 usec, ≤10 usec, ≤5 usec, ≤2.5 usec, or ≤1.5 usec.

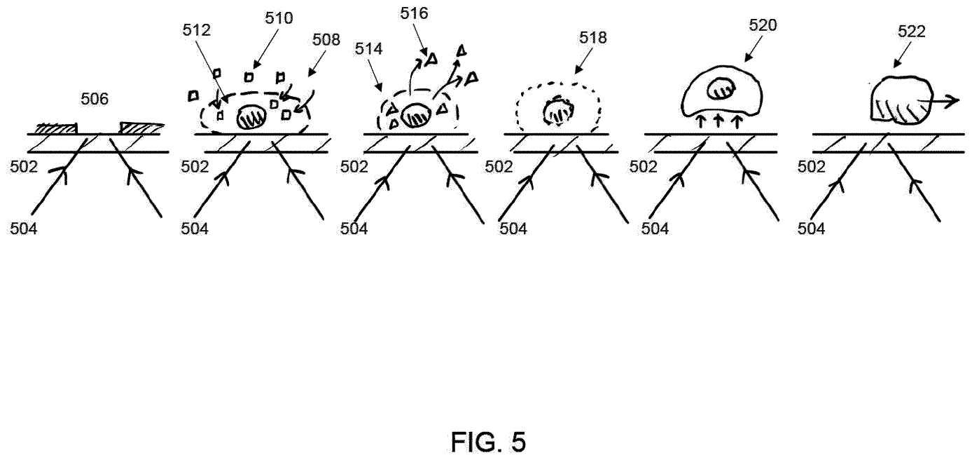

FIG. 5 is a diagram illustrating a number of functions that the physical interaction modes depicted in FIG. 4 may perform in a biological volume in a cell culture container in accordance with various implementations. To create these physical interaction modes, optical radiation 504 impinges on a semitransparent film 502 (e.g., film 302 in FIG. 3). In one example the physical interaction between the optical radiation 504 and the film 502 removes a layer from the surface of the film 502, indicated by gap 506. The layer that may be removed may include, but not be limited to, ECMs, accumulated proteins, various biofilms, films that promote or prevent adhesion of biological materials, films that provide specific biological receptors, or films whose content is subsequently used in the biological volume (for example, reagents, intracellular cargos, factors, nutrients, buffers, etc.).

In another example, the physical interaction between the optical radiation 504 and the film 502 temporarily porates the membrane(s) of one or more cells 508, allowing compounds or cargos 510 in the surrounding medium to enter the cell cytoplasm 512, or even the nuclear envelope. Such intracellular delivery may be enhanced using chemical conditions or differentials (for example, control of calcium levels), use of electric fields, use of liquid flows that apply shear forces to cells, or any combination of the above. In another example, the physical interaction between the optical radiation 504 and the film 502 temporarily porates cell(s) 514 in a manner that allows material 516 within the cells 514 to escape the cell membrane into the surrounding medium. Such material may include, but is not limited to, proteins and enzymes, nucleic acids, lipids, vesicles, exosomes, mitochondria, bacteria or viruses, etc. In another example, the physical interaction between the optical radiation 504 and the film 502 irreversibly porates cell(s) 518 to terminate the cells 518 in the cell culture. Cell material may subsequently be washed off the surface for disposal or analysis of its contents.

In another example, the physical interaction between the optical radiation 504 and the film 502 exerts a force on cell(s) 520 and/or their adhesion points on the film 502 to lift them off the cell culture surface. The lifted cell(s) 520 may subsequently be harvested for downstream processes, or for analysis. They may also be re-adhered within the same cell culture container. In some implementations, certain cell types will de-adhere with the physical interaction, while others will not, such that this may be used as a sorting mechanism. The same physical interaction may be used to detach debris, dead cells, proteins, droplets, temporary microstructures or matrices, or bubbles from the surface. The interaction may be combined with fluidic flows that promote detachment via shear forces and may carry the objects away. In another example, the physical interaction between the optical radiation 504 and the film 502 exerts a force on material 522 in the biological volume, causing its physical translation within the cell culture container. Such forces may be used to clean regions of a cell culture container. They may also be used to sort cells, either by translation in static liquid volumes, or by displacement within flows.

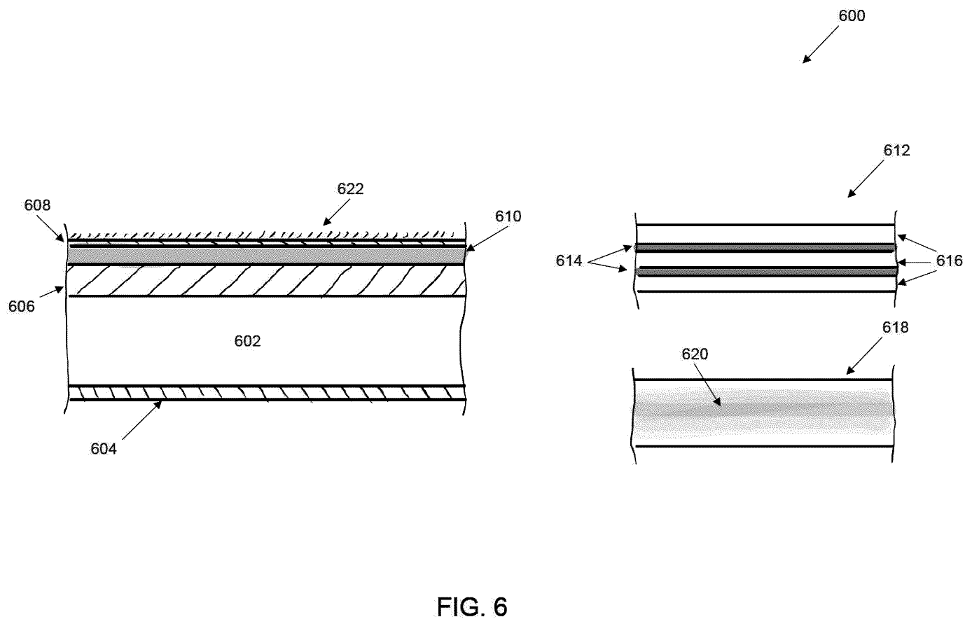

FIG. 6 is a diagram illustrating a biosurface stack 600 for use in a cell culture system in accordance with various implementations. The biosurface stack 600 includes a transparent substrate 602. The substrate 602 may include a number of materials, including but not limited to glasses (including, but not limited to, borosilicate glasses, fused silica, aluminosilicate glasses), polymers (including, but not limited to, polycarbonate, polymethyl methacrylate, cyclo-olefin polymers, cyclo-olefin copolymers, polyimides, fluorinated polyimides), ceramics and glass-ceramics (including, but not limited to, sapphire, spinel, aluminum oxynitride), and crystalline materials (including, but not limited to, calcium fluoride, magnesium fluoride, silicon), composites and hybrids (including, but not limited to, polymer-nanoparticle composites and glass-polymer hybrids).

The bottom surface of the substrate 602 may be coated with an optical layer 604. This optical layer 604 may fulfill optical functions such as acting as an anti-reflection coating for the laser wavelength, imaging wavelengths, or both. In some implementations, the optical layer 604 may provide a certain amount of reflectivity for the purpose of optical sensing, for example by an autofocus system. In such a case, the autofocus system measures light reflected from the back surface/layer, and based on knowledge of the optical thickness of the substrate 602, may adjust focus for the purpose of imaging and/or laser interaction with the absorbing semitransparent films 608, 610 on the surface facing the biological volume. The wavelength for autofocus tracking may be different than that for laser interaction with the semitransparent film, and/or imaging wavelengths used to measure the biological volume. The optical layer 604 may also have other functions, such as to resist scratching and other damage, and potentially to resist condensation or accumulation of debris that could affect imaging and/or laser functions.

The biosurface stack 600 may also include an optional buffer layer 606 that is disposed between the substrate 602 and a semi-absorptive layer 610. This buffer layer 606 may be used to provide an insulating function: if the peak temperatures experienced by the semi-absorptive layer 610 during illumination by optical radiation sources are greater than the maximum temperature sustainable by the substrate material without damage, then the buffer layer 606 may be used to protect the substrate 602 from these high temperatures. For example, if the semi-absorptive layer 610 experiences a peak temperature of 1000° C. for 10 nanoseconds, but the substrate 602 has a maximum temperature before softening of 525° C. (for borosilicate glass), and the goal is to avoid softening where potential mechanical damage can occur, a roughly 240 nm buffer layer of SiO₂ may be used to protect the substrate 602 based on the thermal diffusivity of SiO₂.

The buffer layer 606 may also be incorporated as an optical layer in the overall biosurface stack 600 to optimize reflection, transmission, and absorption characteristics of the stack 600 at various wavelengths, and/or to optimize where within the stack 600 absorption of optical radiation occurs. Finally, the buffer layer 606 may also act as a barrier layer, for oxygen, water, or other species that might affect absorber performance or stability (particularly in the case in which the substrate may be permeable, and/or contain some of these species). Examples of materials that could be used for such a buffer layer 606 include, but are not limited to, aluminum oxide, silicon dioxide, hafnium oxide, yttrium oxide, magnesium fluoride, zirconium oxide, titanium dioxide, and silicon nitride. Materials that are stable at ≥1000° C., at ≥1200° C., at ≥1500° C. or at ≥2000° C. may be used to ensure stability. Materials with melting points of ≥1000° C., of ≥1500° C., or of ≥2000° C. may be used. Materials with low thermal diffusivity, for example ≤1 mm²/sec, ≤2 mm²/sec, or ≤10 mm²/sec may be used to provide good insulation with low film thickness. Materials that have low refractive index, for example ≤1.5, ≤1.8 or ≤2.0 may be used. Materials that have low oxygen diffusivity, for example ≤1 E⁻¹⁷ cm²/sec, ≤1 E⁻¹⁶ cm²/sec, or ≤1 E⁻¹⁵ cm²/sec may be used.

The biosurface stack 600 also includes a semitransparent absorption, or semi-absorptive, layer 610 that serves to absorb optical radiation, heat up, and transmit at least a portion of this resulting thermal energy towards the top surface which faces the biological volume. A wide range of materials may be used as the absorber material. The absorber may be a single layer of material, a multilayer stack consisting of discrete layers, a layer with continuous gradations of material, or combinations of these. Thinner absorbing layers, for example ≤5 nm, ≤10 nm, or ≤20 nm may be used in cases in which the absorbing material and surrounding materials are able to withstand high instantaneous temperatures during exposure to optical radiation, and the absorbance of the material is sufficient to absorb the desired fraction of the optical radiation. Such is the case, for example, with thin layers of metallic materials, including but not limited to gold, silver, titanium, chromium, tungsten, tantalum, hafnium, zirconium, and molybdenum. Additionally, sulfides of some of these elements, including but not limited to MoS₂ and WS₂ may be used. Additionally, metal nitrides include but are not limited to ZrN, TaN, MoN, Mo₂N, WN, TiN, W₂N, and HfN. A number of carbides may also be used as absorbing material, including, but not limited to, HfC, TaC, SiC, TiC, ZrC, NbC, and B₄C. Carbonitride materials may also be used as high temperature stable absorbers, including but not limited to HfCN, TaCN, ZrCN, TiCN, and BCN. Some of these materials are stable to very high peak temperatures in inert environments but oxidize rapidly when oxygen is present, necessitating oxygen barrier layers to be used in or around the light-absorbing layer of the structure.

The biosurface stack 600 may also include a capping layer 608. The capping layer 608, if distinct from the semi-absorptive layer 610, may serve one or more functions including: providing an appropriate, biocompatible, non-cytotoxic surface material facing the biological volume; providing a material surface that has very low dissolution or degradation under the conditions of the biological volume (for example, elevated temperature, saline solution, varying pH range, compounded with the effects of the optical radiation); serving as a barrier that prevents diffusion or intrusion of oxygen, water, or other compounds from the biological volume into the semitransparent absorber; and/or providing a surface that, potentially with biosurface treatment, provides the appropriate level of binding or adsorption for proteins or cells (which may be high binding/adsorption in some cases, and low in other cases). Materials for such a capping layer may include but are not limited to: silicon dioxide, aluminum oxide, zirconium oxide, silicon nitride, magnesium fluoride, titanium aluminum nitride, titanium dioxide, or hafnium oxide. The capping layer 608 may include more than one of these materials, for example, one material (proximate to the semi-absorptive layer 610) that provides a diffusion barrier function, and one material (proximate to the biological volume) that provides the biological interface. The capping layer 608 may be kept thin to minimize thermal diffusion time/thermal resistance when transferring heat from the semi-absorptive layer 610 into the biological volume. For example, it may have a thickness of ≤100 nm, ≤50 nm, or ≤25 nm.

To compose the semi-absorptive layer 610, high-absorbing materials with lower maximum operating temperatures may be distributed in a multilayer absorber stack 612, as described herein, instead of a single absorbing layer 610. The absorber stack 612 may include layers of absorbing materials 614 separated by non-absorbing materials 616. Examples of such absorbing materials 614 include amorphous silicon, amorphous germanium, amorphous silicon-germanium, gold, silver, and others listed herein. Individual layers of these absorbing materials 614 are separated by non-absorbing materials 616 with high temperature capability, including but not limited to silicon dioxide, silicon nitride, aluminum oxide, silicon dioxide, hafnium oxide, yttrium oxide, magnesium fluoride, zirconium oxide, and titanium dioxide. The individual absorbing and non-absorbing layers may be deposited with differing thicknesses to optimize optical and thermal properties of the overall stack.

In some implementations, a graded film 618 may be used, in which deposition techniques allow a continuous tuning of absorption properties, as indicated by gradient 620. An example of this is the use of silicon nitride, in which the nitrogen content is tuned to allow a continuum between amorphous silicon and stoichiometric silicon nitride. The gradient 620 may be symmetrical or non-symmetrical, or periodic.

A biosurface treatment 622 may be applied on top of the biosurface stack 600. Such a treatment serves to modify the top layer of the absorber structure (or the optional capping layer 608, which may also be an oxygen barrier material and/or absorber layer material, depending on the design of the film structure), which is typically inorganic, to withstand high peak temperatures, and to prevent dissolution or leaching of materials into the biological volume. A range of surface treatments or modifications may be used to improve the binding and/or orientation of proteins to support robust attachment and healthy growth of adherent cell types. These proteins may include, but are not limited to, ECM, growth factors, and other binding domains (e.g., RGD peptide). These treatments modify the available chemical groups on the surface of the stack 600 to design a surface which can optimally interact with applied proteins, The surface treatments may control the number of various groups (—COOH, —OH, —NH2, —SO3) on a surface and in effect tune the wettability or charge of the surface to control the protein surface interactions. One type of these surface coatings imparts a greater positive charge to the surface via exposed amines to attract proteins—most of which are negatively charged—via Coulomb forces. The charged surface increases the efficiency of protein adsorption, and may also orient the proteins to have integrin binding sites of these proteins more available to cells. Some coatings also form covalent bonds with electrostatically positioned proteins, for example via mobile electrons following high-energy plasma treatments. Covalent bio-functionalization supports a more durable protein layer than physioadsorption as protein exchange and displacement are avoided.

Numerous methods may be used to increase the positive charge of a film surface. Simple adsorption of polycationic electrolytes (e.g., poly-L-ornithine, poly-L-lysine, polyethyleneimine) may increase the surface charge. A functionally analogous method uses chemical crosslinking to control the application of exposed amines, such as with polyallylamine-grafted-perfluorophenylazide. Yet another method relies on plasma enhanced chemical vapour deposition. By injecting certain gases, such as nitrogen and acetylene, or volatilized monomers, such as diaminopropane, into plasma reactions, thin (sub 10 nm) polymer coatings may be deposited with exposed nitrogenous moieties. Some plasma activated surfaces are rich in free radicals which can covalently immobilize biomolecules following initial electrostatic attraction and positioning.

In some implementations, the buffer layer 606 and the capping layer 608 may be the same material. In some implementations, capping layer 608 and the semi-absorbing layer 610 may be the same material.

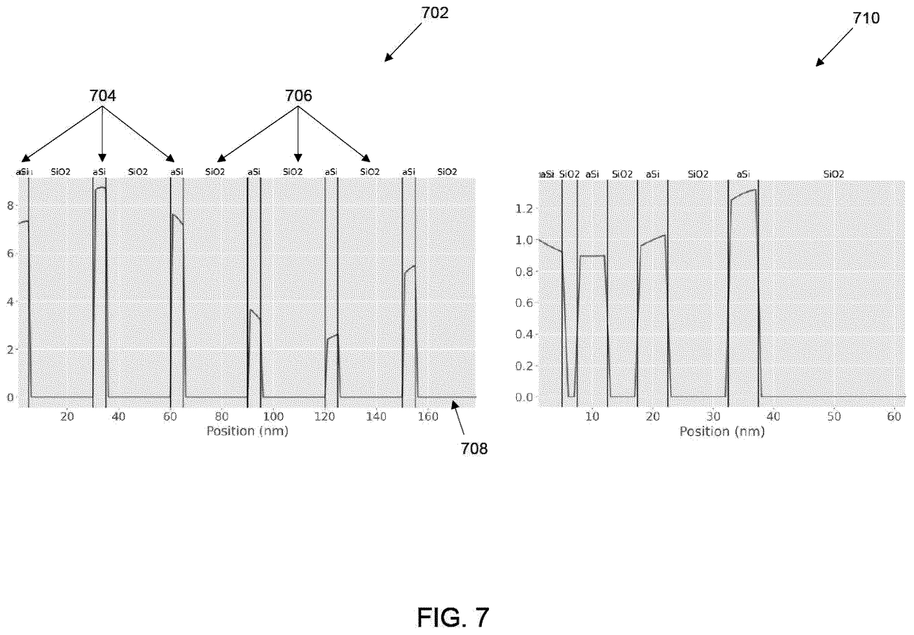

FIG. 7 are diagrams showing examples of tuning of an absorber structure for optical and thermal performance in accordance with various implementations. To tune an absorber structure for a certain level of optical energy absorption, a multilayer absorber structure 702 may be used. The structure 702 may include alternating amorphous silicon absorber layers 704 and non-absorbing silicon dioxide layers 706. The substrate side is to the left of the structure 702, and the biological volume is to the right. When illuminated with 532 nm laser radiation from the substrate side, the resulting absorption of optical energy within the structure is modeled with the curve 708. The varying absorption in the layers results from the optical properties of the entire absorber stack, including both the attenuation due to absorption, but also interference effects, as can be seen by the sinusoidal profile. The result is an undesirable factor of >3× absorption in the second absorbing layer compared to the fifth absorbing layer. The maximum energy before film degradation is therefore limited by the second layer, which also is furthest (spatially, and thermally) from the biological volume.

A revised optical layer design is shown to the right in structure 710, in which non-absorbing SiO₂ layer thicknesses have been adjusted to make the absorption levels in the amorphous silicon layers more uniform. Such modeling may also be used to minimize absorption and transmission variability, and thermal damage threshold variability resulting from thin film manufacturing variability. In addition, such optimization may be used to balance or increase absorption at one wavelength while increasing light transmission at another (for imaging).

Combinations of good optical absorption and high peak temperature capability, enabled by inherent high temperature stability, distributed absorber designs (in which absorption is distributed across multiple absorber layers and therefore peak temperature for a given energy absorption is reduced), and/or oxygen diffusion barrier materials to prevent oxidation of absorbing material at elevated temperature, together may enable thinner structures above the substrate or substrate and buffer layer (if a buffer layer is used). This is advantageous for the purpose of conducting the thermal energy generated from absorption of the optical radiation to the biological volume above the absorber. The overall absorber, buffer layer, and/or biosurface treatment, may for example be ≤500 nm in thickness, ≤200 nm in thickness, ≤100 nm in thickness, ≤75 nm in thickness, ≤50 nm in thickness, ≤30 nm in thickness, or ≤20 nm in thickness. This may enable structures with fast mean thermal diffusion time between the optical absorbing layer and biological volume in which the thermal energy performs its effect, for example ≤500 nsec, ≤250 nsec, ≤100 nsec, or ≤50 nsec.

### Other Considerations

While various implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the implementations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific inventive implementations described herein. It is, therefore, to be understood that the foregoing implementations are presented by way of example only and that, within the scope of the appended claims and equivalents thereto, implementations may be practiced otherwise than as specifically described and claimed. In addition, any combination of two or more such features, systems, aspects, articles, materials, kits, and/or methods, if such features, systems, aspects, articles, materials, kits, and/or methods are not mutually inconsistent, is included within the inventive scope of the present disclosure. Particularly, any element of the disclosure and any aspect thereof may be combined, in any order and any combination, with any other element of the disclosure and any aspect thereof.

The above-described implementations can be implemented in any of numerous ways. For example, the implementations may be implemented using hardware, software or a combination thereof. When implemented in software, the software code can be executed on any suitable processor or collection of processors, whether provided in a single computer or distributed among multiple computers.

As used in any implementation herein, a “circuit” or “circuitry” may include, for example, singly or in any combination, hardwired circuitry, programmable circuitry, state machine circuitry, and/or firmware that stores instructions executed by programmable circuitry. An “integrated circuit” may be a digital, analog or mixed-signal semiconductor device and/or microelectronic device, such as, for example, but not limited to, a semiconductor integrated circuit chip.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device. Also, a computer may have one or more input and output devices. These devices can be used, among other things, to present a user interface. Such computers may be interconnected by one or more networks in any suitable form, including a local area network or a wide area network, such as an enterprise network, an intelligent network (IN) or the Internet. Such networks may be based on any suitable technology and may operate according to any suitable protocol and may include wireless networks, wired networks or fiber optic networks.

The various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

Implementations of the methods described herein may be implemented using a processor and/or other programmable device. To that end, the methods described herein may be implemented on a tangible, non-transitory computer readable medium having instructions stored thereon that when executed by one or more processors perform the methods. The computer readable medium may include any type of tangible medium, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of media suitable for storing electronic instructions.

The terms “program” or “software” are used herein in a generic sense to refer to any type of computer code or set of computer-executable instructions that can be employed to program a computer or other processor to implement various aspects of implementations as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various implementations. Also, data structures may be stored in computer-readable media in any suitable form.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. As used herein in the specification and in the claims, “or” should be understood to have the same meaning as “and/or” as defined above.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “coupled” as used herein refers to any connection, coupling, link or the like by which signals carried by one system element are imparted to the “coupled” element. Such “coupled” devices, or signals and devices, are not necessarily directly connected to one another and may be separated by intermediate components or devices that may manipulate or modify such signals. Likewise, the terms “connected” or “coupled” as used herein in regard to mechanical or physical connections or couplings is a relative term and does not require a direct physical connection.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems.

It will be appreciated by those skilled in the art that any block diagrams herein represent conceptual views of illustrative circuitry embodying the principles of the disclosure. Similarly, it will be appreciated that any flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

We claim:

1. A film stack for optical cell management, comprising.
a substrate having a first surface facing an interior of the cell culture container;
one or more absorbing layers on the first surface of the substrate; and
a biosurface treatment on top of the one or more absorbing layers, wherein cells are adhered to the biosurface treatment.

2. The film stack of claim 1, wherein the stack is configured to transmit optical energy within a first wavelength range to enable optical imaging of cells adhered to the stack.

3. The film stack of claim 2, wherein the stack is configured to absorb optical energy within a second wavelength range to enable optical-based removal of cells adhered to the stack.

4. The film stack of claim 1, wherein the substrate is transparent.

5. The film stack of claim 1, further comprising an optical layer on a second surface of the substrate, the second surface facing an exterior of the cell culture container.

6. The film stack of claim 5, wherein the optical layer comprises at least one of an antireflective coating, a layer that prevents damage to the substrate, and/or a layer that prevents accumulation of debris.

7. The film stack of claim 1, further comprising a buffer layer between the substrate and the one or more absorbing layers.

8. The film stack of claim 7, wherein the buffer layer performs at least one of:
(a) absorbing excess heat from optical energy to prevent damage to the substrate;
(b) tuning reflection, transmission, and/or absorption characteristics of the stack; and/or
(c) acting as a barrier between the substrate and the one or more absorbing layers.

9. The film stack of claim 1, wherein the one or more absorbing layers convert optical energy to mechanical energy or heat energy that is transferred to one or more of the cells adhered to the stack.

10. The film stack of claim 9, wherein the one or more absorbing layers comprise alternating layers of a first material and a second material, wherein the first material absorbs optical energy within a second wavelength range and the second material does not absorb optical energy within the second wavelength range.

11. The film stack of claim 10, wherein the layers of the first material are thinner than the layers of the second material.

12. The film stack of claim 10, wherein the layers of the second material are not of uniform thickness.

13. The film stack of claim 10, wherein the first material comprises at least one of amorphous silicon, amorphous germanium, amorphous silicon-germanium, gold, and silver.

14. The film stack of claim 10, wherein the second material comprises at least one of silicon dioxide, silicon nitride, aluminum oxide, silicon dioxide, hafnium oxide, yttrium oxide, magnesium fluoride, zirconium oxide, and titanium dioxide.

15. The film stack of claim 9, wherein the mechanical energy or heat energy causes the one or more cells adhered to the stack to detach from the biosurface treatment, or to cause sections of the biosurface treatment to detach from the one or more absorbing layers.

16. The film stack of claim 1, further comprising a capping layer on top of the one or more absorbing layers, the capping layer performs at least one of:
(a) serving as a barrier between a biological volume in the cell culture container and the one or more absorbing layers;
(b) aids in adhering cells and/or proteins to the stack;
(c) prevents degradation of the stack into the biological volume; and/or
(d) provides a biocompatible, non-cytotoxic surface exposed to the biological volume.

17. The film stack of claim 1, wherein the biosurface treatment is configured to at least one of encourage cell growth and adhesion, to withstand high temperatures, and to prevent leaching of materials from the stack into a biological volume.

18. The film stack of claim 1, wherein the biosurface treatment comprises an extracellular matrix.

19. A cell culture container comprising a film stack according to claim 1.
